(19) United States
(12) Patent Application Publication
(10) Pub. No.: US 2026/0341458 A1
(43) Pub. Date: Oct. 29, 2026

(54) MACHINE LEARNING FOR MODALITY SELECTION

(71) Applicant: Brightree LLC, Peachtree Corners, GA (US)

(72) Inventor: Jaimini Sankhavaram PANINI, Atlanta, GA (US)

(21) Appl. No.: 19/638,383

(22) Filed: Apr. 3, 2026

Related U.S. Application Data
(60) Provisional application No. 63/794,090, filed on Apr. 24, 2025.

(51) Int. Cl. G16H 20/30

(52) U.S. Cl. CPC G16H 20/30

(57) ABSTRACT

Techniques for improved machine learning are provided. An uncompleted action of a user is identified, the uncompleted action relating to a respiratory therapy of the user. A plurality of attributes of the user is determined, and a request modality is selected based on processing the plurality of attributes using one or more machine learning models. Using the selected request modality, it is requested that the user perform the uncompleted action in connection with the respiratory therapy.

### CROSS-REFERENCE TO RELATED APPLICATIONS

This application claims benefit of co-pending United States provisional patent application Serial No. 63/794,090 filed Apr. 24, 2025. The aforementioned related patent application is herein incorporated by reference in its entirety.

### TECHNICAL FIELD

The present disclosure relates generally to machine learning, and more particularly, to use of machine learning to improve request delivery.

In many systems, a wide variety of communications are provided to users, including reminders, requests, instructions, and the like. In some environments, it is beneficial or necessary for the user to complete one or more specified actions, where prompt action completion is generally desirable. Although reminders and requests can be sent to attempt to expedite such completion, use of such reminders can incur substantial expense (including computationally and physically), often with minimal (or no) benefit accrued.

### SUMMARY

According to some implementations of the present disclosure, a method includes: identifying an uncompleted action of a user, the uncompleted action relating to a respiratory therapy of the user; determining a plurality of attributes of the user; selecting, based on processing the plurality of attributes using one or more machine learning models, a request modality; and requesting, using the selected request modality, that the user perform the uncompleted action in connection with the respiratory therapy.

According to some implementations of the present disclosure, a system includes a control system and a memory. The control system includes one or more processors. The memory has stored thereon machine readable instructions. The control system is coupled to the memory, and any one of the methods disclosed herein is implemented when the machine executable instructions in the memory are executed by at least one of the one or more processors of the control system.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective embodiments.

FIG. 1 depicts an example system for improved machine learning-based request generation, according to some embodiments of the present disclosure.

FIG. 2 depicts an example system for training machine learning models for improved request generation, according to some embodiments of the present disclosure.

FIG. 3 depicts an example workflow for improved machine learning-based request generation, according to some embodiments of the present disclosure.

FIG. 4 depicts an example workflow for training machine learning models for improved request generation, according to some embodiments of the present disclosure.

FIG. 5 is a flow diagram depicting an example method for training machine learning models to facilitate request generation, according to some embodiments of the present disclosure.

FIG. 6 is a flow diagram depicting an example method for using machine learning models to facilitate request generation, according to some embodiments of the present disclosure.

FIG. 7 is a flow diagram depicting an example method for scoring modalities, times, and days for request generation, according to some embodiments of the present disclosure.

FIG. 8 is a flow diagram depicting an example method for request generation, according to some embodiments of the present disclosure.

FIG. 9 depicts an example computing device configured to perform various document ingestion aspects of the present disclosure, according to some embodiments disclosed herein.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure generally provide techniques for using machine learning for improved request generation.

In many environments, it is desirable for users to complete indicated actions in a timely manner, such as to expedite service initialization and/or ensure continuity. For example, in some therapeutic settings (e.g., for users who engage in respiratory therapy, such as using a continuous positive airway pressure (CPAP) system), prompt user response in completing actions such as requesting or finalizing resupply (e.g., for consumable components such as air conduits), signing or approving documents, and/or paying for services or items in connection with the therapy is beneficial. In some cases, messages may be communicated to the user to facilitate or expedite such action. For example, reminders or requests may be provided to prompt the user to perform the action. Although respiratory therapy is used as one example environment in which embodiments of the present disclosure may be used, the disclosed embodiments are readily applicable to a wide variety of other therapies, such as therapy and/or equipment for diabetes, cardiovascular therapies, and the like.

However, many such messages do not successfully prompt the user to perform the action. For example, depending on the timing and modality of the request, many users may ignore the message, defer responding or performing the action until a later time (which risks the user forgetting to complete it later), or may not even receive the communication (e.g., due to timing mismatch). As a result, a large portion of such requests do not achieve the desired goal. Further, many such requests incur substantial expense in preparation and transmission, depending on the particular implementation. For example, requests sent via electronic mail (e.g., email) and/or application notifications (e.g., push notifications by an app on the user’s smartphone) can consume substantial network bandwidth, potentially causing congestion and harming network stability and throughput (particularly when large batches of such requests are sent at once). Similarly, requests sent via text message cause additional congestion on cellular networks. As another example, requests sent via physical mail (e.g., a letter) consume significant physical resources both in creating the communication (e.g., consuming paper) as well as in delivering it (e.g., consuming petroleum or other products to transport the letter).

Because a large portion of these requests do not actually prompt user action, these resources are often wasted. In some embodiments of the present disclosure, to reduce or prevent wasted computational and/or physical resource expense, machine learning model(s) may be trained and used to predict the optimal (or at least improved) transmission attributes for such requests. For example, model(s) may be trained to predict the best modality (e.g., email, text, push notification, or physical mail), the best time (e.g., what time of day the request should be sent and/or delivered) and/or the best day of the week (e.g., the day when the request should be sent and/or delivered) for the specific recipient user. Using such predictions can improve the probability that any given request will be successful, which can substantially reduce computational expense and wasted resources.

### Example System for Improved Machine Learning-Based Request Generation

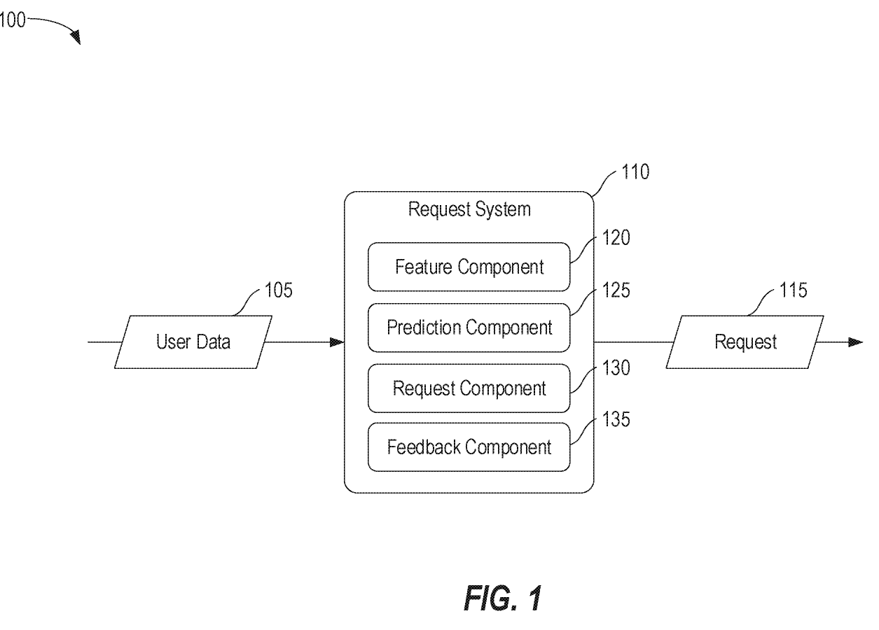

FIG. 1 depicts an example system 100 for improved machine learning-based request generation, according to some embodiments of the present disclosure.

In the illustrated system 100, user data 105 is accessed by a request system 110 to generate a request 115. As used herein, “accessing” data may generally include receiving, retrieving, requesting, obtaining, collecting, generating, or otherwise gaining access to the data. For example, the request system 110 may retrieve the user data 105 from one or more storage repositories (which may include repositories local to the request system 110), or may receive the user data 105 from another application or entity (e.g., from a user that is triggering the request 115). Although depicted as a discrete system for conceptual clarity, in some embodiments, the functionality of the request system 110 may be performed individually and/or jointly by any number of devices and systems, such as in a cloud deployment.

In some embodiments, the user data 105 generally includes information about one or more user attributes that can be evaluated to generate an optimal (or at least improved) request 115. For example, in some embodiments, the user data 105 can include user demographics, such as the user’s age, gender, location (e.g., country, state, city, and/or zip code), and the like. In some embodiments, the user data 105 is accessed in response to determining (e.g., by the request system 110) that a request 115 should be generated for the user (e.g., in response to determining that there are one or more uncompleted actions associated with the user).

In the illustrated example, the request system 110 includes a feature component 120, a prediction component 125, a request component 130, and a feedback component 135. Although depicted as discrete components for conceptual clarity, in some embodiments, the operations of the depicted components (and others not illustrated) may be combined or distributed across any number and variety of components, and may be implemented using hardware, software, or a combination of hardware and software.

In some embodiments, the feature component 120 may be used to extract and/or generate features based on the user data 105. For example, given a set of information about the user, the feature component 120 may generate machine learning features for the relevant data, such as by determining the corresponding information (e.g., computing the user’s age or determining the user’s listed location), converting the information to another format to facilitate machine learning (e.g., converting a text string to a numerical vector), and the like. In some embodiments, the particular features (and feature formatting) generated by the feature component 120 may vary depending on the particular implementation, such as depending on the particular features that the model(s) have been trained to use as input.

In some embodiments, the prediction component 125 may be used to generate or select optimal (or at least improved) requests, such as by selecting a modality, time, and/or day for sending the request 115. In some embodiments, the prediction component 125 may select the transmission characteristics by processing the user attributes (e.g., the features generated by the feature component 120) using one or more trained machine learning models. Generally, the particular architectures used may vary depending on the particular implementation.

For example, in some embodiments, the prediction component 125 may use a first model to generate a selected modality, a second model to generate a select time, and a third model to generate a selected day. As another example, the prediction component 125 may use a first model to select the modality and a second model to select the day and time. As another example, the prediction component 125 may use a single model to select the modality, day, and time. In some embodiments, the prediction component 125 may use a different model depending on the action that is desired from the user. That is, the prediction component 125 may determine what action is being requested (e.g., paying for an order, completing a resupply request, signing a document, providing information, and the like) and may generate the output predictions using a corresponding model that was trained specifically for the determined action. Use of such action-specific models may enable the models to each learn the particular features and patterns that are relevant to the given action (e.g., because the optimal transmission parameters may vary depending on the particular action that is requested).

In some embodiments, to generate the transmission parameters, the prediction component 125 may generate a set of scores for a set of alternative options. For example, the prediction component 125 may generate a respective modality score for each respective modality (of a set of alternative modalities). The prediction component 125 can then select the highest-scored modality to use for the request 115.

In some embodiments, the request component 130 may generate, transmit, or otherwise facilitate the request 115 using the generated transmission parameters. For example, if the selected modality is a digital means (e.g., email, text message, push notification, and the like) the request component 130 may itself generate and transmit the request 115 in accordance with the generated parameters (e.g., transmitting the request at the indicated time on the indicated day using the indicated modality). If the selected modality is a physical means (e.g., mailing a letter), the request component 130 may facilitate the communication using various means, such as by triggering the letter to be printed by one or more printing devices, creating a record indicating the transmission parameters (e.g., indicating the date and/or time when the letter should be mailed), and the like.

In some embodiments, the feedback component 135 can monitor the request 115 and/or the user’s response to the request 115 to refine the machine learning model(s) used by the prediction component 125. For example, after the request 115 is transmitted, the feedback component 135 may monitor the user actions to determine whether the user completes the requested action (e.g., at all, or within a defined window, such as within a week, or prior to a second request being sent). If so, the feedback component 135 may evaluate criteria such as the time that elapsed between the request 115 being sent by the request system 110 and/or received by the user and the time when the action was completed. In some embodiments, longer delays before completing the action may correlate to lower scores generated by the models (e.g., such that the request system 110 is less likely to use the same transmission parameters in the future, given the same set of input user attributes).

In some embodiments, the feedback component 135 may determine whether the user used a link to complete the action. For example, the request 115 may include a link or code (e.g., a hyperlink that the user can click to follow, a code that the user can enter (e.g., via typing), and the like, in order to facilitate completion of the action. In some embodiments, when the action is completed, the feedback component 135 can determine whether this link was used (e.g., based on parameters embedded in the link, based on the code provided by the user, and the like) to determine whether the request 115 prompted the action. If the link or code was not used, in some embodiments, the feedback component 135 may determine that the user acted independently. In some embodiments, in addition to or instead of evaluating whether a link or code was used, the feedback component 135 may compare the action completion with the time when the request 115 was received or delivered. For example, if the user completed the action within minutes of receiving the request 115, the feedback component 135 may infer that the request prompted the action (even if the user did not use the link or code).

That is, using a generated link or code (or comparing the completion time and the receive time of the request), the feedback component 135 may determine whether the action completion can be attributed to the request 115. In some embodiments, if the completion is attributed to the request (e.g., the feedback component 135 infers that the request prompted the action), such as because the user used the embedded link or entered the provided code, the feedback component 135 may generate a new training exemplar for the request 115. This exemplar can then be used to refine the models (e.g., based on the delay until the user completed the action), as discussed in more detail below. In some embodiments, if the action cannot be confidently attributed to the request 115 (e.g., because the user did not use the embedded link, or because a relatively larger period of time has elapsed before the action was completed), the feedback component 135 may refrain from refining the models based on this request, or may attribute the action to another request (e.g., the request that included the link used by the user, or a request that did not include a link).

For example, if the feedback component 135 determines that the user did not use a link or code, the feedback component 135 may identify the most recent request, sent to the user, which did not include a link. The feedback component 135 may then infer that this most-recent request (without a link) prompted the action.

Generally, refining the machine learning model(s) may include a variety of operations. For example, the feedback component 135 may use the user attributes (e.g., the features from the user data 105) as the input portion of the exemplar (e.g., used as input to the model). Further, a score may be generated based on the user’s action completion (e.g., where longer delays correlate to a smaller score), and the output prediction can then be compared against the score (e.g., where larger differences between the predicted score for the selected transmission parameters and the actual score generated based on the user’s completion result in a larger loss). This loss can then be used to refine the model. For example, if the feedback component 135 determines that the user delayed a relatively long time before completing the action, the feedback component 135 may generate a large loss and refine the model parameters (e.g., using backpropagation) such that, given the same set of input user attributes, the models will be less likely to generate the transmission parameter(s) that were used for the request 115. Similarly, if the feedback component 135 determines that the user performed the action quickly after receiving the request 115, the feedback component 135 may generate a smaller loss and refine the model parameters such that, given the same set of input user attributes, the models will be more likely to generate the transmission parameter(s) that were used for the request 115.

In some embodiments, by using machine learning models to generate the transmission parameters, the request system 110 may substantially reduce the computational and physical expense of providing requests to users, as discussed above. Further, the use of machine learning can improve the probability that the user completes the action, resulting in a variety of corresponding benefits including improved therapeutic results and service continuity.

### Example System for Training Machine Learning Models for Improved Request Generation

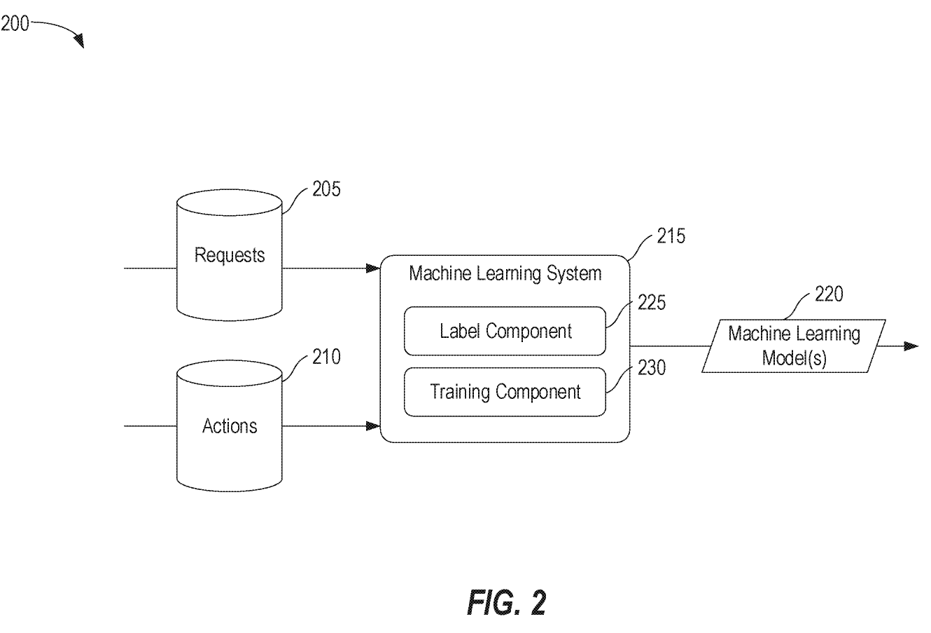

FIG. 2 depicts an example system 200 for training machine learning models for improved request generation, according to some embodiments of the present disclosure.

In the illustrated system 1200, requests 205 and actions 210 are accessed by a machine learning system 215 to generate or train one or more machine learning models 220. Although depicted as a discrete system for conceptual clarity, in some embodiments, the functionality of the machine learning system 215 may be performed individually and/or jointly by any number of devices and systems, such as in a cloud deployment. In some embodiments, the machine learning system 215 may correspond to (e.g., be implemented by the same computing system as) the request system 110 of FIG. 1.

In some embodiments, the requests 205 generally includes information about one or more prior requests (e.g., the request 115 of FIG. 1) that were sent to users, asking the users to complete one or more specified actions. In some embodiments, each record in the requests 205 includes information relating to a specific request, such as the transmission parameters used (e.g., the modality, time, and day), the user to whom the request was targeted (e.g., the user attributes of the user), and the like.

In some embodiments, the actions 210 include information about actions completed by one or more users (which may or may not have been prompted by a request). For example, each record in the actions 210 may include information such as what action was completed, when the action was completed, whether the user used a link, code, or other specified identifier when completing the action, and the like.

In the illustrated example, the machine learning system 215 includes a label component 225 and a training component 230. Although depicted as discrete components for conceptual clarity, in some embodiments, the operations of the depicted components (and others not illustrated) may be combined or distributed across any number and variety of components, and may be implemented using hardware, software, or a combination of hardware and software.

In some embodiments, the label component 225 may evaluate the requests 205 and/or actions 210 to determine or infer, for each respective request 205, how successful the request was (e.g., whether the request caused the user to complete the action, how much time elapsed between the action and the request, and the like). In some embodiments, the label component 225 may additionally or alternatively evaluate the actions 210 to determine or infer, for each action 210, the corresponding request 205 that prompted the action 210 (if any), the delay between receiving the request and completing the action, and the like.

In some embodiments, if the label component 225 is able to attribute the action 210 to a request 205, the label component 225 can generate a training exemplar (e.g., including the user data as the exemplar’s input) and generating a label for the exemplar based on factors such as the delay between the user receiving the request and completing the action (or whether the request prompted the action at all). In some embodiments, the generated score may be associated with the transmission parameters that were used for the request. For example, if the parameters resulted in a long delay before completing the action (or did not prompt the completion at all, such as if the user only completed the action after another request or without using the link included in the request), the label component 225 may assign a low score to the selected modality, time, and/or day. Using this low score can allow the model to be trained to generate a low score for the given transmission parameters in the future, given the same inputs.

In some embodiments, the training component 230 may use the generated exemplars to train the one or more machine learning models 220. Generally, the particular operations used to train the models may vary depending on the particular architecture(s) used. For example, in the case of a neural network, the training component 230 may process the exemplar’s input (e.g., the user attributes) using the model to generate a set of score(s) (e.g., one score for each alternative modality). The training component 230 can then compare the generated score with the actual score (e.g., generated by the label component 225), and refine the model based on the difference (e.g., using backpropagation).

In this way, the training component 230 can train machine learning models 220 to generate improved requests in the future. For example, the training component 230 will learn to generate higher scores for transmission parameters that improve the probability of quick action completion (based on the particular user attributes), while generating lower scores for transmission parameters that are less likely to result in quick action.

As illustrated, after training, the machine learning models 220 may be deployed for runtime use. As used herein, “deploying” the models may include a wide variety of actions to prepare or provide the models for use, depending on the particular implementation. For example, the machine learning system 215 may serialize the parameters and transmit them to an inferencing system (e.g., the request system 110 of FIG. 1) for use. As another example, if the machine learning system 215 itself generates requests, the machine learning system 215 may instantiate the model locally for runtime use.

### Example Workflow for Improved Machine Learning-Based Request Generation

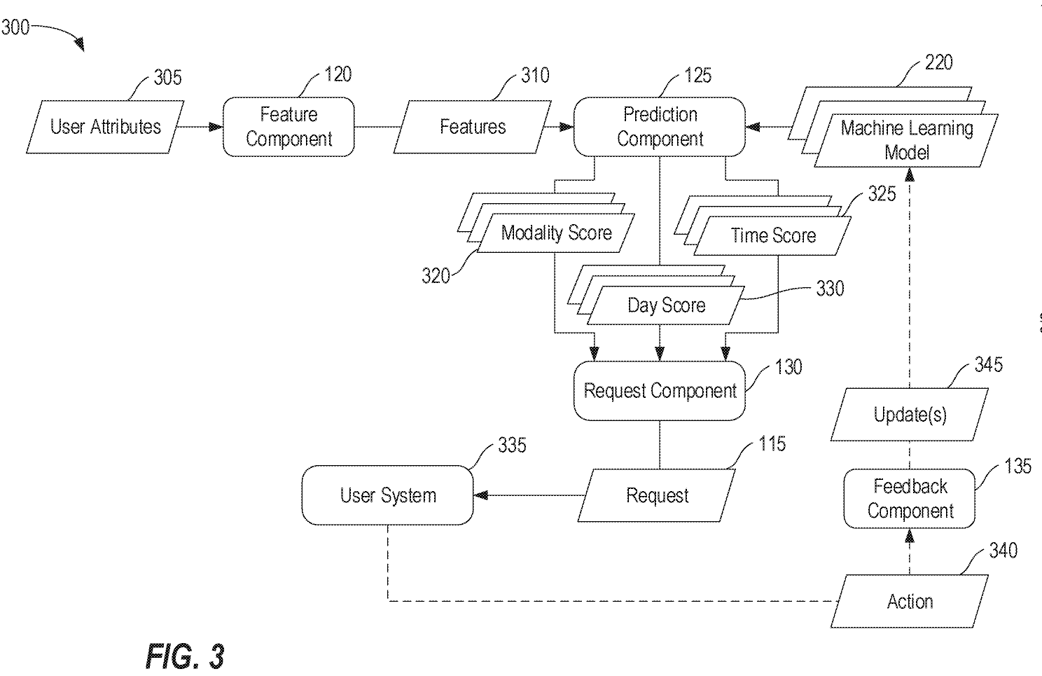

FIG. 3 depicts an example workflow 300 for improved machine learning-based request generation, according to some embodiments of the present disclosure. In some embodiments, the workflow 300 is performed by a request system, such as the request system 110 of FIG. 1.

As illustrated, a set of user attributes 305 (e.g., corresponding to the user data 105 of FIG. 1) is accessed by the feature component 120. As discussed above, the user attributes 305 can generally include any relevant information for a user for whom a request is being generated, such as the user’s demographics.

In the illustrated workflow 300, based on the user attributes 305, the feature component 120 generates a set of feature 310. Generally, the particular contents and format of the features 310 may vary depending on the particular implementation. For example, the feature component 120 may generate one feature 310 (such as the age of the user) based on the current date and the user’s birthdate. In some embodiments, the feature component 120 may generate a numerical feature encoding the user’s location (e.g., city, state, and/or zip code) based on the user attributes 305.

As illustrated, the features 310 are then processed by the prediction component 125, using one or more machine learning models 220, to generate a corresponding set of output scores, including a set of modality scores 320, a set of time scores 325, and a set of day scores 330.

In some embodiments, the modality scores 320 include a respective modality score for each respective modality of a set of alternative modalities that can be used to provide the request. For example, the modality scores 320 may include a first score for a “text message” modality, a second score for an “email” modality, and so on. In some embodiments, the modality scores 320 are each a continuous value in a defined range (e.g., between zero and one), where higher scores indicate a higher preference for the given modality (e.g., a higher predicted probability that the corresponding modality will result in quick user action). In some embodiments, the prediction component 125 generates the modality scores 320 by processing the features 310 using a classifier model trained to generate, for each respective class (e.g., each respective modality), a score indicating the probability that the given modality is the optimal or best option. In this way, the model learns to select the best modality for the given set of user attributes.

In some embodiments, the time scores 325 similarly include a respective time score for each respective time of a set of alternative times at which the request can be transmitted and/or delivered. In some embodiments, the alternative times can be classified into categories, such as “morning,” “afternoon,” and “evening,” or by ranges of time such as “9:00-12:00,” “12:00-15:00,” and the like. For example, the time scores 325 may include a first score for a first time window, a second score for a second time window, and so on. In some embodiments, as discussed above with reference to the modality scores 320, the time scores 325 are each a continuous value in a defined range (e.g., between zero and one), where higher scores indicate a higher preference for the given time window (e.g., a higher predicted probability that transmitting and/or delivering the request during the corresponding window will result in quick user action). In some embodiments, the prediction component 125 generates the time scores 325 by processing the features 310 using a classifier model trained to generate, for each respective class (e.g., each window of time), a score indicating the probability that the given time is the optimal or best option. In this way, the model learns to select the best transmission time for the given set of user attributes.

In some embodiments, the day scores 330 similarly include a respective day score for each respective day of a set of alternative days during which the request can be transmitted and/or delivered (e.g., each day of the week). For example, the day scores 330 may include a first score for a first day (e.g., Monday), a second score for a second day (e.g., Tuesday), and so on. In some embodiments, as discussed above with reference to the modality scores 320 and time scores 325, the day scores 330 are each a continuous value in a defined range (e.g., between zero and one), where higher scores indicate a higher preference for the given day (e.g., a higher predicted probability that transmitting and/or delivering the request during the corresponding day will result in quick user action). In some embodiments, the prediction component 125 generates the day scores 330 by processing the features 310 using a classifier model trained to generate, for each respective class (e.g., each day of the week), a score indicating the probability that the given day is the optimal or best option. In this way, the model learns to select the best transmission day for the given set of user attributes.

In some embodiments, the prediction component 125 generates the modality scores 320, the time scores 325, and the day scores 330 using a single machine learning model. In some embodiments, the prediction component 125 uses a respective machine learning model for each respective set of scores (e.g., a first model to generate the modality scores 320, a second model to generate the time scores 325, and a third model to generate the day scores 330). In some embodiments, the prediction component 125 uses action-agnostic models (e.g., where a single model may be trained to generate scores regardless of what action the user is being requested to perform). In some embodiments, the prediction component 125 uses action-specific models (e.g., where each model may be trained to generate scores indicating the best request parameters if the corresponding specific action is desired).

Although the illustrated example depicts a set of three specific request parameters (e.g., the modality, time, and day) for conceptual clarity, in some embodiments, the prediction component 125 may generate scores for a wide variety of transmission or request parameters, depending on the particular implementation.

In the illustrated example, the modality scores 320, time scores 325, and day scores 330 are accessed and evaluated by the request component 130 to generate the request 115 for the user. For example, as discussed above, the request component 130 may facilitate transmission of the request 115 using the selected modality (e.g., the modality having the highest modality score 320) at the selected time (e.g., the window of time having the highest time score 325) and on the selected day (e.g., the day having the highest day score 330).

In the illustrated example, the request 115 is provided to a user system 335 (e.g., a computing device associated with or belonging to the user, such as the user’s smartphone, laptop, desktop computer, tablet, and the like). In some embodiments¸ the particular method of delivery depends on the selected modality, as discussed above. Although the illustrated example depicts the request 115 being provided to the user system 335, in some embodiments, the request 115 may be provided by other means or to other destinations (e.g., using physical mail).

In the illustrated workflow 300, as depicted by the dashed arrow, the user may then use the user system 335 to complete the requested action 340. For example, if the request 115 included a link or code (e.g., a hyperlink or universal resource locator (URL) including a code identifying the request 115), the user may use the user system 335 to follow the link and perform the action 340 (e.g., completing payment, requesting resupply, signing the documentation, and the like).

In the illustrated example, the action 340 may be evaluated by the feedback component 135 to generate update(s) 345 to the machine earning models 220. For example, as discussed above, the feedback component 135 may determine which request 115, if any, the action 340 can be attributed to (e.g., based on the indicated code). The updates 345 may then be used to refine the machine learning models 220 based on the determination that the identified request 115 prompted the user to perform the action 340. In some embodiments, as discussed above, this update 345 may be further based on the time that elapsed between the request 115 and the action 340. In some embodiments, for requests 115 that did not prompt action, the feedback component 135 can similarly generate updates 345 that cause the corresponding transmission parameters to receive lower scores fur subsequent iterations.

In these ways, as discussed above, the request system can significantly reduce the computational expense and network congestion of the system (e.g., by reducing the number of requests 115 that are generated and transmitted) while simultaneously improving the therapeutic experience of the user (e.g., because relevant actions are completed in a timely manner to prevent service interruption or degradation).

### Example Workflow for Training Machine Learning Models for Improved Request Generation

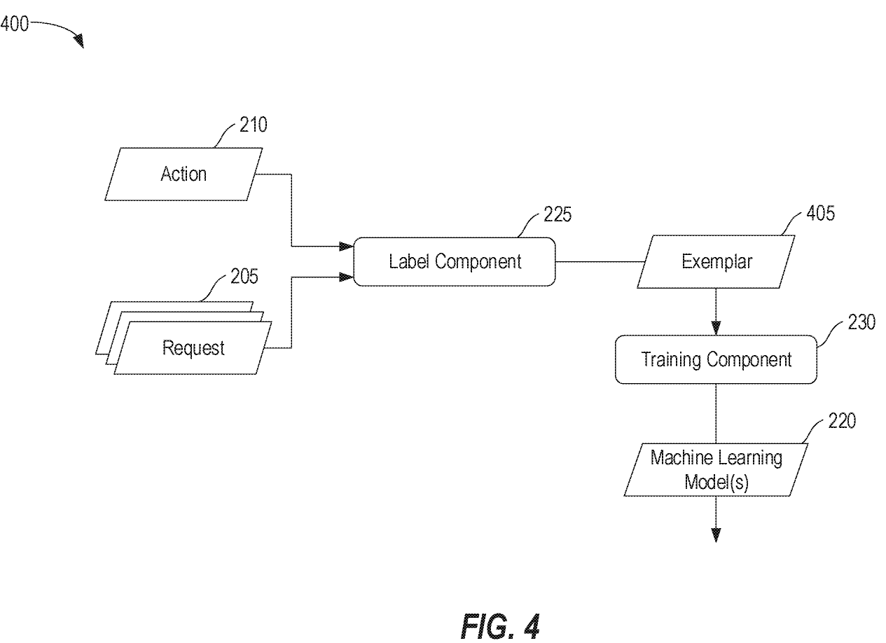

FIG. 4 depicts an example workflow 400 for training machine learning models for improved request generation, according to some embodiments of the present disclosure. In some embodiments, the workflow 400 is performed by a machine learning system, such as the machine learning system 215 of FIG. 2.

In the illustrated workflow 400, an action 210 (e.g., a historical record of an action completed previously by a user, such as signing a document, requesting resupply, and the like) and a set of requests 205 (e.g., one or more requests that were sent to the user, asking the user to perform the action 210) are accessed by the label component 225. As discussed above, based on these inputs, the label component 225 can generate one or more exemplars 405.

In some embodiments, to generate the exemplar 405, the label component 225 may attribute the action 210 to one or more specific requests 205. For example, if the action 210 was completed using a link or code embedded in a request 205, the label component 225 can attribute the action 210 to this request 205. In some embodiments, if the action 210 was not completed using a link or code, the label component 225 can attribute the action 210 to the most recent request 205, or to the most recent request that did not include such a link or code. In some embodiments, if a defined period of time has elapsed between the action 210 and the request 205, the label component 225 may determine that none of the requests are responsible for prompting the action 210.

In some embodiments, the label component 225 generates an exemplar for the request 205 that prompted the action 210 (e.g., the request to which the action is attributed by the label component 225). For example, as discussed above, the label component 225 may generate an exemplar 405 that includes the user attributes as the input portion, with a label indicating the transmission parameters used by the request 205 (e.g., the modality, time, and/or day) that prompted the action. In some embodiments, as discussed above, the label may further include or be generated based on a score for the action 210, such as based on the time that elapsed before the user completed the action (e.g., where higher delays result in lower scores).

In some embodiments, the label component 225 may generate one or more exemplars 405 for any requests 205 that did not prompt the action 210 (e.g., requests to which the action cannot be attributed). For example, as discussed above, the label component 225 may generate an exemplar 405 that includes the user attributes as the input portion, with a label indicating that the transmission parameters used by the request 205 (e.g., the modality, time, and/or day) failed to prompt the desired action (at all, or within a desired time period).

As illustrated, these exemplar(s) 405 can then be used by the training component 230 to generate, train, refine, fine-tune, or otherwise modify the machine learning model(s) 220 used to generate the request parameters. For example, as discussed above, the training component 230 may process the input portion of the exemplar 405 to generate output scores, and may then generate a loss by comparing these scores with the label(s) generated by the labeling component 225. This loss can then be used to refine the machine learning models 220, iteratively refining the parameters (e.g., weights and biases) to allow the machine learning models 220 to learn to predict more accurate output (e.g., scores indicating which transmission parameters are most likely to prompt quick action, given the user attributes).0

As discussed above, the machine learning models 220 can then be deployed for runtime use.

### Example Method for Training Machine Learning Models to Facilitate Request Generation

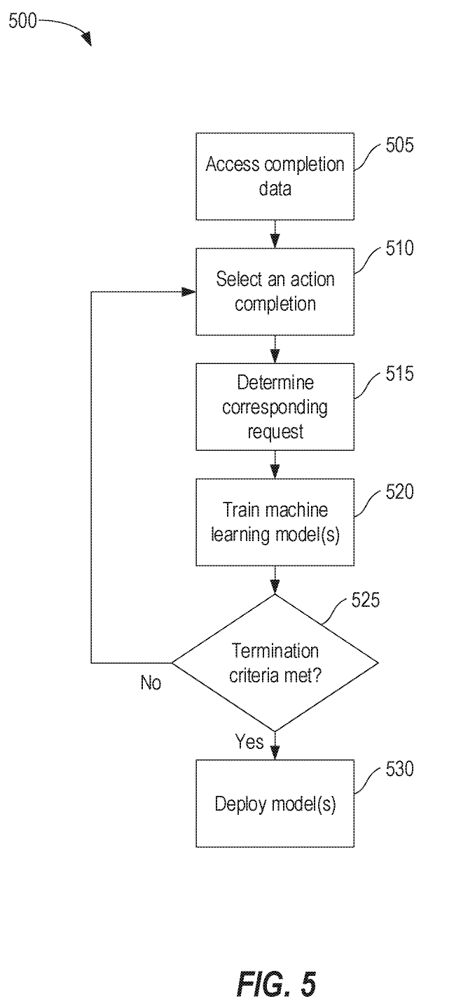

FIG. 5 is a flow diagram depicting an example method 500 for training machine learning models to facilitate request generation, according to some embodiments of the present disclosure. In some embodiments, the method 500 is performed by a computing system, such as a machine learning system (e.g., the machine learning system 215 of FIG. 2) and/or a request system (e.g., the request system 110 of FIG. 1).

At block 505, the computing system accesses a set of action completion data (e.g., the actions 210 of FIGS. 2 and /or 4 ) indicating the completion of a requested action in connection with respiratory therapy of a user. For example, the action completion data may include records indicating when one or more users completed one or more requested or desired actions, such as ordering resupply, completing a payment, signing a document, providing requested information, and the like. Generally, the completion data may include information for any number of actions performed by any number of users.

At block 510, the computing system selects one of the action completion records (e.g., the action 210 of FIG. 4) from the completion data. Generally, the computing system may use a variety of techniques to select the action, including randomly or pseudo-randomly, as the computing system may evaluate all of the records during the method 500.

At block 515, the computing system determines the corresponding request that prompted the selected action. That is, the computing system may determine the request (e.g., the request 205 of FIG. 4) to which the completed action can be attributed. For example, in some embodiments, the computing system may determine which code or link the user used when completing the action (e.g., determining which request included or provided the code or link that was used). As another example, in some embodiments, the computing system may determine the most recent request that was received, by the user, prior to the user completing the action.

At block 520, the computing system trains one or more machine learning model(s) (e.g., the machine learning models 220 of FIGS. 2 and /or 4 ) based on the selected action and corresponding request. For example, as discussed above, the computing system may train the model by indicating that the transmission parameters associated with the request (e.g., the time, day, and/or modality) resulted in the user performing the action (e.g., by using the transmission parameters as the target output or label for the model, given the set of user attributes determined for the user to whom the request was transmitted).

At block 525, the computing system determines whether one or more termination criteria are met, with respect to the training. For example, the computing system may determine whether one or more training exemplars remain, whether the model has reached a desired level of accuracy, and the like. Although the illustrated example depicts training the model using a single selected action (e.g., using stochastic gradient descent) for conceptual clarity, in some embodiments, the computing system may additionally or alternatively train the model based on multiple exemplars in parallel (e.g., using batch gradient descent).

In the termination criteria are not met, the method 500 returns to block 510. If the criteria are met, the method 500 continues to block 530, where the computing system deploys the machine learning model(s) for runtime use, as discussed above.

### Example Method for Using Machine Learning Models to Facilitate Request Generation

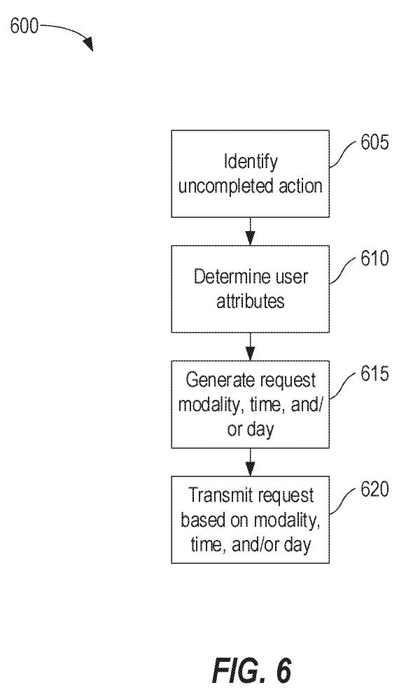

FIG. 6 is a flow diagram depicting an example method 600 for using machine learning models to facilitate request generation, according to some embodiments of the present disclosure. In some embodiments, the method 600 is performed by a computing system, such as a request system (e.g., the request system 110 of FIG. 1) and/or a machine learning system (e.g., the machine learning system 215 of FIG. 2).

At block 605, the computing system identifies an uncompleted action. That is, the computing system determines that at least one action (e.g., in connection with a respiratory therapy of a user), as not been completed by the user. For example, the computing system may determine that the user should request resupply (e.g., because it the consumable items are expecting to wear out soon), that the user should complete payment for previous services, that user approval or signature is needed for one or more items, and the like.

At block 610, the computing system determines a set of user attributes (e.g., the user data 105 of FIG. 1 and/or the user attributes 305 of FIG. 3) for the identified user (e.g., demographic information such as the user’s age, gender, location, and the like).

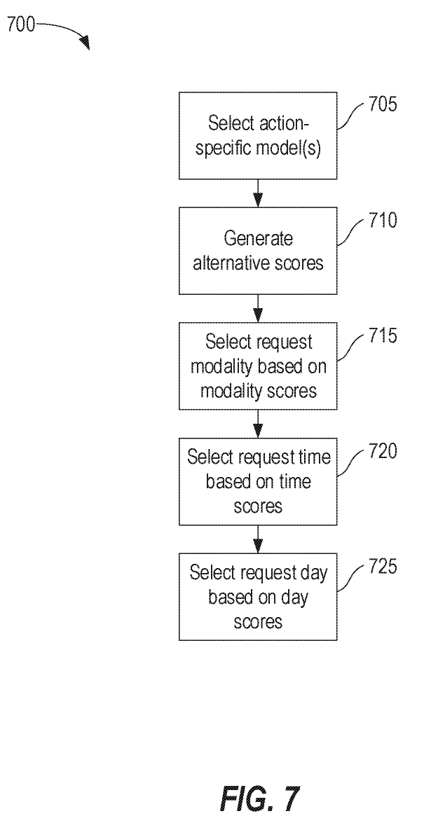

At block 615, the computing system generates a set of request parameters, such as a request modality, a request time, and/or a request day based on processing the user attributes (e.g., features extracted from user data) using one or more trained machine learning models (e.g., the machine learning models 220 of FIG. 3). In some embodiments, as discussed above, the computing system may use action-specific model(s) (e.g., where the model(s) used to generate the request parameters were trained specifically based on the uncompleted action, such as using a first set of models to generate output for “documentation” actions, a second set of models to generate output for “resupply” actions, and the like). In some embodiments, the computing system may use action-agnostic models. One example, method for generating the request parameters is discussed in more detail below with reference to FIG. 7.

At block 620, the computing system can transmit a request (e.g., the request 115 of FIGS. 1 and/ or 3 ) to the user (e.g., via the user system 335 of FIG. 3) based on or in accordance with the generated modality, time, and/or day. For example, as discussed above, the computing system may transmit or sent the request (or cause the request to be transmitted or sent) using the indicated modality at the indicated time on the indicated day (or may send the request such that it will be received at the indicated time on the indicated day).

As discussed above, this machine learning-based request targeting can significantly improve the probability that a given request will prompt action, thereby reducing the number of such requests that are used. This reduction in requests results in significant reductions in computational expense and network traffic.

### Example Method for Scoring Modalities, Times, and Days for Request Generation

FIG. 7 is a flow diagram depicting an example method 700 for scoring modalities, times, and days for request generation, according to some embodiments of the present disclosure. In some embodiments, the method 700 is performed by a computing system, such as a request system (e.g., the request system 110 of FIG. 1) and/or a machine learning system (e.g., the machine learning system 215 of FIG. 2). In some embodiments, the method 700 provides additional detail for block 615 of FIG. 6.

At block 705, the computing system selects one or more action-specific models based on the action(s) desired. For example, if the uncompleted action relates to resupply, the computing system may select the model(s) trained to improve resupply requests. Although the illustrated example depicts selection of action-specific models, in some embodiments, the computing system may use action-agnostic models, as discussed above.

At block 710, the computing system generates a set of alternative scores, using the selected model(s), for the various transmission alternatives that can be used. For example, as discussed above, the computing system may generate a respective modality score for each alternative modality, a respective time score for each alternative window of time, and/or a respective day score for each alternative day.

At block 715, the computing system selects a request modality based on the modality scores (e.g., the modality scores 320 of FIG. 3). For example, as discussed above, the computing system may select the modality having the highest modality score (e.g., because this modality is predicted to be the most likely to cause the user to complete the action, and/or the modality that is predicted to cause the user to complete the action most quickly).

At block 720, the computing system selects a request time based on the time scores (e.g., the time scores 325 of FIG. 3). For example, as discussed above, the computing system may select the time having the highest time score (e.g., because this window of time is predicted to be the most likely to cause the user to complete the action, and/or the time window that is predicted to cause the user to complete the action most quickly).

At block 730, the computing system selects a request day based on the day scores (e.g., the day scores 330 of FIG. 3). For example, as discussed above, the computing system may select the day having the highest day score (e.g., because this day is predicted to be the most likely to cause the user to complete the action, and/or the day that is predicted to cause the user to complete the action most quickly).

In these ways, as discussed above, the computing system can significantly improve the probability that the request will prompt action, thereby reducing the number of such requests that are used. This reduction in requests results in significant reductions in computational expense and network traffic.

### Example Method for Request Generation

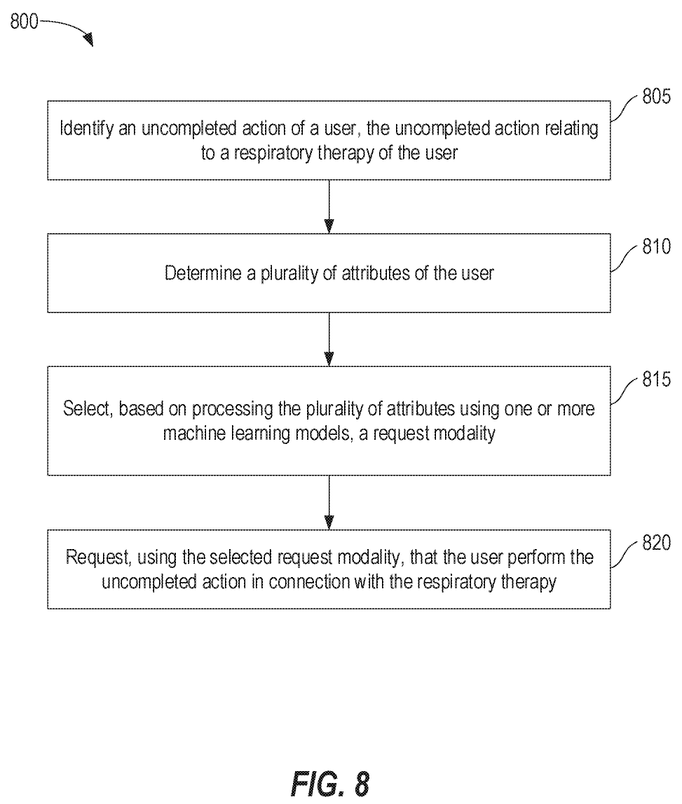

FIG. 8 is a flow diagram depicting an example method 800 for request generation, according to some embodiments of the present disclosure. In some embodiments, the method 800 is performed by a computing system, such as a request system (e.g., the request system 110 of FIG. 1) and/or a machine learning system (e.g., the machine learning system 215 of FIG. 2).

At block 805, an uncompleted action of a user is identified, the uncompleted action relating to a respiratory therapy of the user.

At block 810, a plurality of attributes (e.g., the user attributes 305 of FIG. 3) of the user is determined.

At block 815, a request modality is selected, based on processing the plurality of attributes using one or more machine learning models (e.g., the machine learning models 220 of FIG. 3).

At block 820, using the selected request modality, it is requested that the user perform the uncompleted action in connection with the respiratory therapy.

### Example Processing System for Request Generation

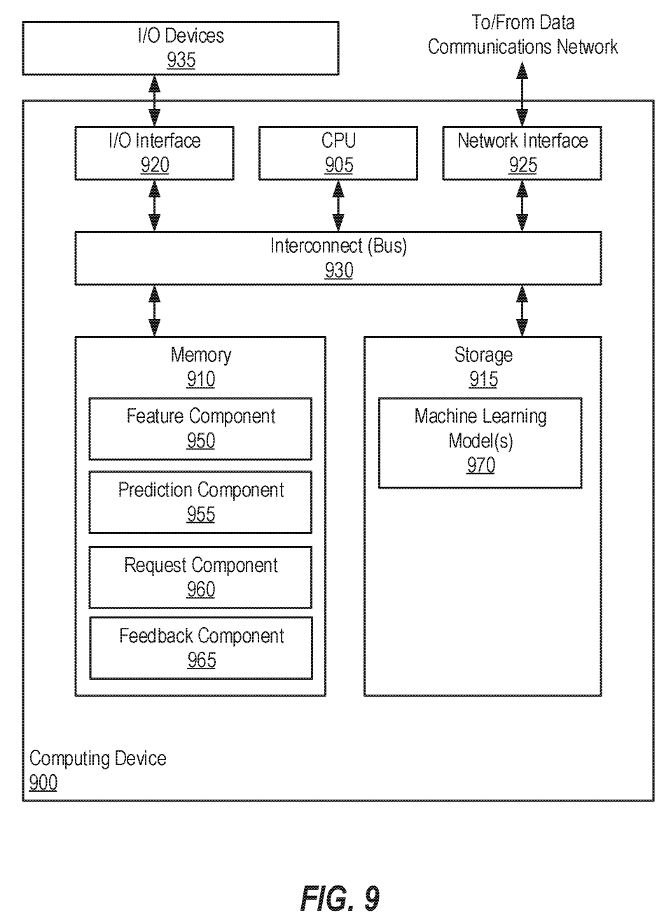

FIG. 9 depicts an example computing device 900 configured to perform various aspects of the present disclosure, according to some embodiments disclosed herein. Although depicted as a physical device, in embodiments, the computing device 900 may be implemented using virtual device(s), and/or across a number of devices (e.g., in a cloud environment). In one embodiment, the computing device 900 corresponds to a request system (e.g., the request system 110 of FIG. 1) and/or a machine learning system (e.g., the machine learning system 215 of FIG. 2).

As illustrated, the computing device 900 includes a CPU 905, memory 910, storage 915, a network interface 925, and one or more input/output (I/O) interfaces 920. In the illustrated embodiment, the CPU 905 retrieves and executes programming instructions stored in memory 910, as well as stores and retrieves application data residing in storage 915. The CPU 905 is generally representative of a single CPU and/or GPU, multiple CPUs and/or GPUs, a single CPU and/or GPU having multiple processing cores, and the like. The memory 910 is generally included to be representative of a random access memory. Storage 915 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 935 (such as keyboards, monitors, etc.) are connected via the I/O interface(s) 920. Further, via the network interface 925, the computing device 900 can be communicatively coupled with one or more other devices and components (e.g., via a network, which may include the Internet, local network(s), and the like). As illustrated, the CPU 905, memory 910, storage 915, network interface(s) 925, and I/O interface(s) 920 are communicatively coupled by one or more buses 930.

In the illustrated embodiment, the memory 910 includes a feature component 950, a prediction component 955, a request component 960, and a feedback component 965, which may perform one or more embodiments discussed above. Although depicted as discrete components for conceptual clarity, in embodiments, the operations of the depicted components (and others not illustrated) may be combined or distributed across any number of components. Further, although depicted as software residing in memory 910, in embodiments, the operations of the depicted components (and others not illustrated) may be implemented using hardware, software, or a combination of hardware and software.

In some embodiments, the feature component 950 (which may correspond to the feature component 120 of FIGS. 1 and /or 3 ) can be used to extract or generate user attributes (e.g., the feature 310 of FIG. 3), as discussed above. For example, the feature component 950 may features for user demographics, such as the user’s age, location, and the like.

In some embodiments, the prediction component 955 (which may correspond to the prediction component 125 of FIGS. 1 and /or 3 ) can be used to generate predictions relating to transmission alternatives, as discussed above. For example, the prediction component 955 may use machine learning models (e.g., the machine learning models 970) to generate scores (e.g., the modality scores 320, the time scores 325, and/or the day scores 330) indicating the probably that a given transmission alternative (e.g., a given modality) will result in the user completing a requested action (or will result in the fasted action completion, as compared to the alternatives).

In some embodiments, the request component 960 (which may correspond to the request component 130 of FIGS. 1 and /or 3 ) can be used to generate user requests (e.g., the request 115 of FIGS. 1 and /or 3 ), as discussed above. For example, the request component 960 generate and/or transmit the requests in accordance with the selections generated by the prediction component 955 (e.g., at the indicated time on the indicated day and using the indicated modality).

In some embodiments, the feedback component 965 (which may correspond to the feedback component 135 of FIGS. 1 and /or 3 ) can be used to refine the machine learning model(s), as discussed above. For example, after a request is provided to a user, the feedback component 965 may monitor user actions to determine whether and when the user completest he requested action (and, in some cases, whether the request caused the action). This feedback can be used to refine the models to generate improved predictions.

In the illustrated example, the storage 915 includes a set of machine learning models 970 (which may correspond to the machine learning models 220 of FIGS. 2-3). Although not depicted in the illustrated example, in some embodiments, the storage 915 may also include other data such as training data. Although depicted as residing in storage 915, the depicted data may be stored in any suitable location, including memory 910.

Generally, the depicted components (and others not depicted) in memory 910 may evaluate and/or use the depicted data (and others not depicted) in storage 915 to provide accurate and efficient request evaluations, as discussed above.

### Example Clauses

Clause 1: A method, comprising: identifying an uncompleted action of a user, the uncompleted action relating to a respiratory therapy of the user; determining a plurality of attributes of the user; selecting, based on processing the plurality of attributes using one or more machine learning models, a request modality; and requesting, using the selected request modality, that the user perform the uncompleted action in connection with the respiratory therapy.

Clause 2: A method according to Clause 1, further comprising: selecting, based on processing the plurality of attributes using the one or more machine learning models, a request time; and selecting, based on processing the plurality of attributes using the one or more machine learning models, a request day, wherein requesting that the user perform the uncompleted action is performed at the request time on the request day.

Clause 3: A method according to any of Clauses 1-2, wherein the request modality is of a plurality of request modalities comprising at least one of: (i) text message, (ii) electronic mail, (iii) application notification, or (iv) physical mail.

Clause 4: A method according to any of Clauses 1-3, wherein the uncompleted action comprises at least one of: (i) requesting resupply of one or more components of the respiratory therapy, (ii) requesting payment in connection with the respiratory therapy, or (iii) requesting a signature from the user.

Clause 5: A method according to any of Clauses 1-4, wherein selecting the request modality comprises selecting a first machine learning model, of the one or more machine learning models, trained based on a type of the uncompleted action.

Clause 6: A method according to any of Clauses 1-5, wherein the plurality of attributes comprise demographics information for the user.

Clause 7: A method according to any of Clauses 1-6, wherein selecting the request modality comprises generating, for each respective request modality of a plurality of request modalities, a respective score using the one or more machine learning models.

Clause 8: A method according to any of Clauses 1-7, further comprising: determining that the user completed the uncompleted action; identifying a request that prompted the user to complete the uncompleted action; and refining the one or more machine learning models based on the identified request.

Clause 9: A method according to Clause 8, wherein identifying the request that prompted the user to complete the uncompleted action comprises: determining a link that the user used to complete the uncompleted action; and determining that the request included the link.

Clause 10: A method according to Clause 8, wherein identifying the request that prompted the user to complete the uncompleted action comprises: determining that the user did not use a link to complete the uncompleted action; and determining that the request is a most recent request that did not include a link.

Clause 11: Any patentable subject matter described herein.

Clause 12: A processing system, comprising: one or more processors; and one or more memories collectively comprising computer-executable instructions which, when executed on any combination of the one or more processors, cause the processing system to perform a method in accordance with any one of Clauses 1-10.

Clause 13: A system, comprising means for performing a method in accordance with any one of Clauses 1-10.

Clause 14: One or more non-transitory computer readable media collectively containing, in any combination, computer program code that, when executed by operation of a computing system, causes the computing system to perform a method in accordance with any one of Clauses 1-10.

Clause 15: A computer program product embodied on a computer-readable storage medium comprising code for performing a method in accordance with any one of Clauses 1-10.

### Additional Considerations

One or more elements or aspects or steps, or any portion(s) thereof, from one or more of any of claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present invention, a user may access applications or systems (e.g., the document ingestion system) or related data available in the cloud. For example, the document ingestion system could execute on a computing system in the cloud and perform automated document ingestion. In such a case, the system could receive and process raw documents, and store the resulting structured documents in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
identifying an uncompleted action of a user, the uncompleted action relating to a respiratory therapy of the user;
determining a plurality of attributes of the user;
selecting, based on processing the plurality of attributes using one or more machine learning models, a request modality; and
requesting, using the selected request modality, that the user perform the uncompleted action in connection with the respiratory therapy.

2. The method of claim 1, further comprising:
selecting, based on processing the plurality of attributes using the one or more machine learning models, a request time; and
selecting, based on processing the plurality of attributes using the one or more machine learning models, a request day, wherein requesting that the user perform the uncompleted action is performed at the request time on the request day.

3. The method of claim 1, wherein the request modality is of a plurality of request modalities comprising at least one of: (i) text message, (ii) electronic mail, (iii) application notification, or (iv) physical mail.

4. The method of claim 1, wherein the uncompleted action comprises at least one of: (i) requesting resupply of one or more components of the respiratory therapy, (ii) requesting payment in connection with the respiratory therapy, or (iii) requesting a signature from the user.

5. The method of claim 1, wherein selecting the request modality comprises selecting a first machine learning model, of the one or more machine learning models, trained based on a type of the uncompleted action.

6. The method of claim 1, wherein the plurality of attributes comprise demographics information for the user.

7. The method of claim 1, wherein selecting the request modality comprises generating, for each respective request modality of a plurality of request modalities, a respective score using the one or more machine learning models.

8. The method of claim 1, further comprising:
determining that the user completed the uncompleted action;
identifying a request that prompted the user to complete the uncompleted action; and
refining the one or more machine learning models based on the identified request.

9. The method of claim 8, wherein identifying the request that prompted the user to complete the uncompleted action comprises:
determining a link that the user used to complete the uncompleted action; and
determining that the request included the link.

10. The method of claim 8, wherein identifying the request that prompted the user to complete the uncompleted action comprises:
determining that the user did not use a link to complete the uncompleted action; and
determining that the request is a most recent request that did not include a link.

11. A processing system, comprising:
one or more processors; and
one or more memories collectively comprising computer-executable instructions which, when executed on any combination of the one or more processors, cause the processing system to perform an operation comprising:
identifying an uncompleted action of a user, the uncompleted action relating to a respiratory therapy of the user;
determining a plurality of attributes of the user;
selecting, based on processing the plurality of attributes using one or more machine learning models, a request modality; and
requesting, using the selected request modality, that the user perform the uncompleted action in connection with the respiratory therapy.

12. The processing system of claim 11, the operation further comprising:
selecting, based on processing the plurality of attributes using the one or more machine learning models, a request time; and
selecting, based on processing the plurality of attributes using the one or more machine learning models, a request day, wherein requesting that the user perform the uncompleted action is performed at the request time on the request day.

13. The processing system of claim 11, wherein the request modality is of a plurality of request modalities comprising at least one of: (i) text message, (ii) electronic mail, (iii) application notification, or (iv) physical mail.

14. The processing system of claim 11, wherein the uncompleted action comprises at least one of: (i) requesting resupply of one or more components of the respiratory therapy, (ii) requesting payment in connection with the respiratory therapy, or (iii) requesting a signature from the user.

15. The processing system of claim 11, the operation further comprising:
determining that the user completed the uncompleted action;
identifying a request that prompted the user to complete the uncompleted action, comprising:
determining a link that the user used to complete the uncompleted action; and
determining that the request included the link; and
refining the one or more machine learning models based on the identified request.

16. One or more non-transitory computer readable media collectively containing, in any combination, computer program code that, when executed by operation of a computing system, causes the computing system to perform an operation comprising:
identifying an uncompleted action of a user, the uncompleted action relating to a respiratory therapy of the user;
determining a plurality of attributes of the user;
selecting, based on processing the plurality of attributes using one or more machine learning models, a request modality; and
requesting, using the selected request modality, that the user perform the uncompleted action in connection with the respiratory therapy.

17. The one or more non-transitory computer readable media of claim 16, the operation further comprising:
selecting, based on processing the plurality of attributes using the one or more machine learning models, a request time; and
selecting, based on processing the plurality of attributes using the one or more machine learning models, a request day, wherein requesting that the user perform the uncompleted action is performed at the request time on the request day.

18. The one or more non-transitory computer readable media of claim 16, wherein the request modality is of a plurality of request modalities comprising at least one of: (i) text message, (ii) electronic mail, (iii) application notification, or (iv) physical mail.

19. The one or more non-transitory computer readable media of claim 16, wherein the uncompleted action comprises at least one of: (i) requesting resupply of one or more components of the respiratory therapy, (ii) requesting payment in connection with the respiratory therapy, or (iii) requesting a signature from the user.

20. The one or more non-transitory computer readable media of claim 16, the operation further comprising:
determining that the user completed the uncompleted action;
identifying a request that prompted the user to complete the uncompleted action, comprising:
determining a link that the user used to complete the uncompleted action; and
determining that the request included the link; and
refining the one or more machine learning models based on the identified request.
